(19) United States
(12) Patent Application Publication
(10) Pub. No.: US 2003/0120036 A1
(43) Pub. Date: Jun. 26, 2003

(54) Human NOC2-related gene variants associated with lung cancer

(75) Inventors: Ken-Shwo Dai, Hsinchu (TW); Chin-Chang Yang, Hsinchu (TW)

Correspondence Address: Ladas & Parry, 26 West 61st Street, New York, NY, 10023, US

(73) Assignee: BIOPTIK TECHNOLOGY, INC.

(21) Appl. No.: 09/964,275

(22) Filed: Sep. 26, 2001

(51) Int. Cl. C07K 14/47; C07H 21/04; C12P 21/02; C12N 5/06

(52) U.S. Cl. 530/350; 536/23.5; 435/69.1; 435/320.1; 435/325

(57) ABSTRACT

The invention relates to the nucleic acid and polypeptide sequences of five novel human NOC2-related gene variants.

The invention also provides a process for producing the polypeptides of the variants.

The invention fierier provides a use of the nucleic acid and polypeptide sequences of the gene variants in diagnosing non-small cell lung cancer (NSCLC), in particular, large cell lung cancer.

### FIELD OF THE INVENTION

The invention relates to the nucleic acid and polypeptide sequences of five novel human NOC2-related gene variants, preparation process thereof, and uses of the same in diagnosing non-small cell lung cancer (NSCLC), in particular, large cell lung cancer.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4: 292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggested that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes associated with the chromosomal regions frequently altered in lung cancers. For NSCLC, alterations have been documented on chromosomes 3p, 11p and 17p (Ihde and Minna, (1991) Cancer 15: 105-54). On chromosome 17p, mutation of the p53 gene, a tumor suppressor gene, was reported to be associated with NSCLC (Kohno et al. (1999) Cancer 85: 341-7). Recently, a novel tumor suppressor gene, NOC2 (also named RPH3AL), isolated as a human ortholog of rat NOC2 gene, was found to be located on chromosome 17p (Smith et al. (1999) Genomics 59: 97-101).

Rat NOC2 gene was isolated from a rat islet cDNA library under a low stringency hybridization conditions using a mouse rabphilin-3A cDNA as a probe. Sequence analysis demonstrated that a cysteine-rich zinc finger domain was conserved on both NOC2 and rabphilin-3A. The cysteine-rich zinc finger domain of NOC2 has been shown to be a protein-protein interaction interface which links NOC2 to Zyxin (a cytoskeletal element) through an interaction of this domain with the LIM domain of Zyxin (Kotake et al. (1997) J Biol Chem 272: 29407-10). NOC2 was also reported to interact with Rab3A by serving as a direct inhibitor on Rab3A-, associated Ca²⁺-regulated exocytosis (Haynes et al. (2001) J Biol Chem 276: 9726-32). Rab3A is a low-molecular-weight guanosine triphosphate (GTP)-binding protein expressed at high levels in neuronal presynaptic terminals and functionally associated with vesicle transport and Ca²⁺-dependent exocytosis, particularly in the secretion of neurotransmitters (Geppert et al. (1994) Nature 369: 493-7; Oishi et al. (1998) J Biol Chem 273: 34580-5). It is interesting to note that Rab3 family members are essential for cell division since perturbations of Rab3-protein interactions lead to cessation of the cell division (Conner and Wessel, (2000) FASEB J 14:1559-66). In addition, high expression level of Rab3A gene has been found in cancers (Culine et al. (1992) Cancer 70: 2552-6). The presence of Rab3A-Rabphilin3A complex in cancers (Araki et al. (2000) Pigment Cell Res 13: 332-6) suggests that Rab3A-NOC2 may play a role in cancers in addition to a role in Ca²⁺-regulated exocytosis (Haynes et al. (2001) J Biol Chem 276: 9726-32) since NOC2 is structurally related to Rabphilin3A⁻ (Kotake et al. (1997) J Biol Chem 272: 29407-10). Together with chromosomal localization of NOC2, it is believed that NOC2 may be involved in NSCLC.

### SUMMARY OF THE INVENTION

The present invention provides five NOC2 variants present in human lung tissues. The nucleotide sequences of these variants and polypeptide sequences encoded thereby can be used for the diagnosis of any diseases associated with these variants or NSCLC, in particular, the large cell lung cancer.

The invention further provides an expression vector and host cell for expressing the variants.

The invention further provides a method for producing the variants.

The invention further provides an antibody specifically binding to the variants.

The invention also provides methods for detecting the presence of the variants in a mammal.

### BRIEF DESCRIPTION OF THE DRAWINGS

FIG. 1 shows the nucleic acid sequence (SEQ ID NO:1) and amino acid sequence (SEQ ID NO:2) of NL1.

FIG. 2 shows the nucleic acid sequence (SEQ ID NO:3) and amino acid sequence (SEQ ID NO:4) of LC1.

FIG. 3 shows the nucleic acid sequence (SEQ ID NO:5) and amino acid sequence (SEQ ID NO:6) of LC2.

FIG. 4 shows the nucleic acid sequence (SEQ ID NO:7) and amino acid sequence (SEQ ID NO:8) of LC3.

FIG. 5 shows the nucleic acid sequence (SEQ ID NO:9) and amino acid sequence (SEQ ID NO:10) of LC4.

FIG. 6 shows the nucleotide sequence alignment between the human NOC2 gene and its related gene variants (NL1 and LC1 to LC4).

FIG. 7 shows the amino acid sequence alignment between the human NOC2 protein and its related polypeptide variants (NL1 and LC1 to LC4).

### DETAILED DESCRIPTION OF THE INVENTION

According to the present invention, all technical and scientific terms used have the same meanings as commonly understood by persons skilled in the art.

The term “antibody” used herein denotes intact molecules (a polypeptide or group of polypeptides) as well as fragments thereof, such as Fab, R(ab′)₂, and Fv fragments, which are capable of binding the epitopic determinant. Antibodies are produced by specialized B cells after stimulation by an antigen. Structurally, antibody consists of four subunits including two heavy chains and two light chains. The internal surface shape and charge distribution of the antibody binding domain is complementary to the features of an antigen. Thus, antibody can specifically act against the antigen in an immune response.

The term “base pair (bp)” used herein denotes nucleotides composed of a purine on one strand of DNA which can be hydrogen bonded to a pyrimidine on the other strand. Thymine (or uracil) and adenine residues are lined by two hydrogen bonds. Cytosine and guanine residues are lined by three hydrogen bonds.

The term “Basic Local Alignment Search Tool (BLAST; Altschul et al., (1997) Nucleic Acids Res. 25: 3389-3402)” used herein denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a, nucleotide query sequence against the six-frame translations of a nucleotide sequence database.

The term “cDNA” used herein denotes nucleic acids that synthesized from a m-RNA template using reverse transcriptase,

The term “cDNA library” used herein denotes a library composed of complementary DNAs which are reverse-transcribed from mRNAs.

The term “complement” used herein denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion” used herein denotes a removal of a portion of one or more amino acid residues/nucleotides from a gene.

The term “expressed sequence tags (ESTs)” used herein denotes short (200 to 500 base pairs) nucleotide sequence that derives from either 5′ or 3′ end of a cDNA.

The term “expression vector” used herein denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell” used herein denotes a cell which is used to receive, maintain, and allow the reproduction of an expression vector comprising DNA. Host cells are transformed or transfected with suitable, vectors constructed using recombinant DNA methods. The recombinant DNA introduced with the vector is replicated whenever the cell divides.

The term “insertion” or “addition” used herein denotes the addition of a portion of one or more amino acid residues/nucleotides to a gene.

The term “in silico” used herein denotes a process of using computational methods (e.g., BLAST) to analyze DNA sequences.

The term “polymerase chain reaction (PCR)” used herein denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide” used herein denotes a sequence of amino acids in a specific order that can be encoded by a gene or by a recombinant DNA. It can also be chemically synthesized.

The term “nucleic acid sequence” or “polynucleotide” used herein denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR)” used herein denotes a process which transcribes mRNA to complementary DNA strand using reverse transcriptase followed by polymerase chain reaction to amplify the specific fragment of DNA sequences.

The term “transformation” used herein denotes a process describing the uptake, incorporation, and expression of exogenous DNA by prokaryotic host cells.

The term “transfection” used herein a process describing the uptake, incorporation, and expression of exogenous DNA by eukaryotic host cells.

The term “variant” used herein denotes a fragment of sequence (nucleotide or amino acid) inserted or deleted by one or more nucleotides/amino acids.

According to the present invention, the polypeptides of five novel human NOC2-related gene variants and fragments thereof, and the nucleic acid sequences encoding the same are provided.

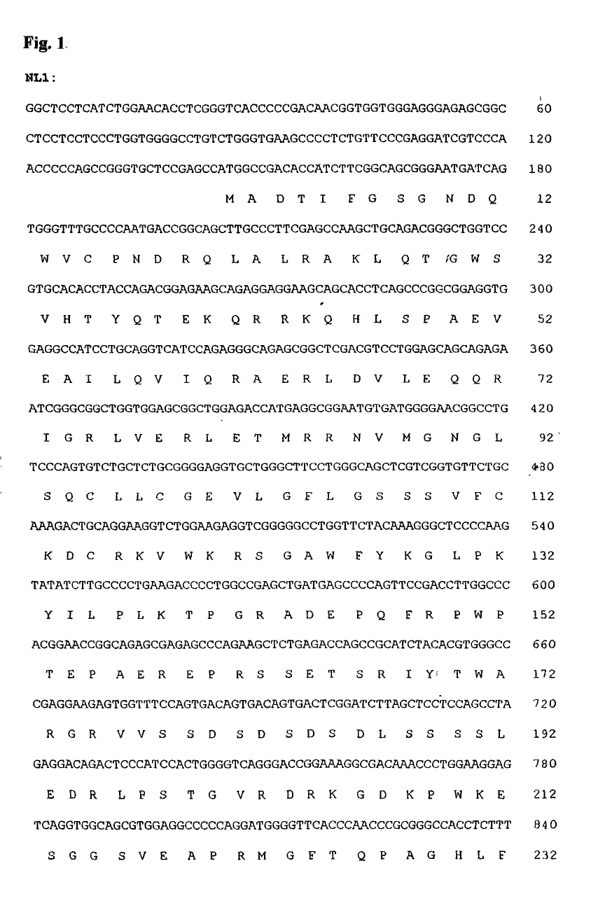

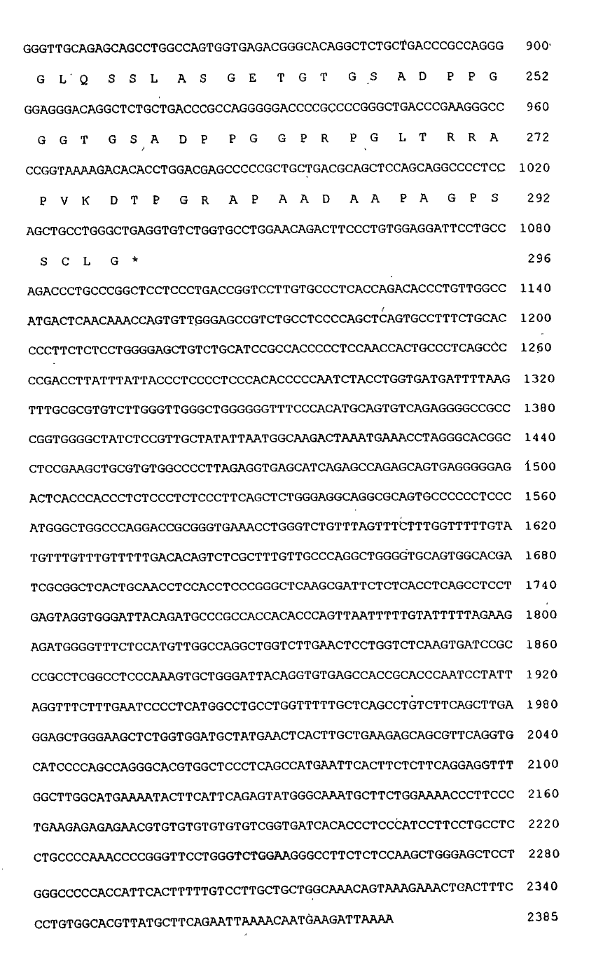

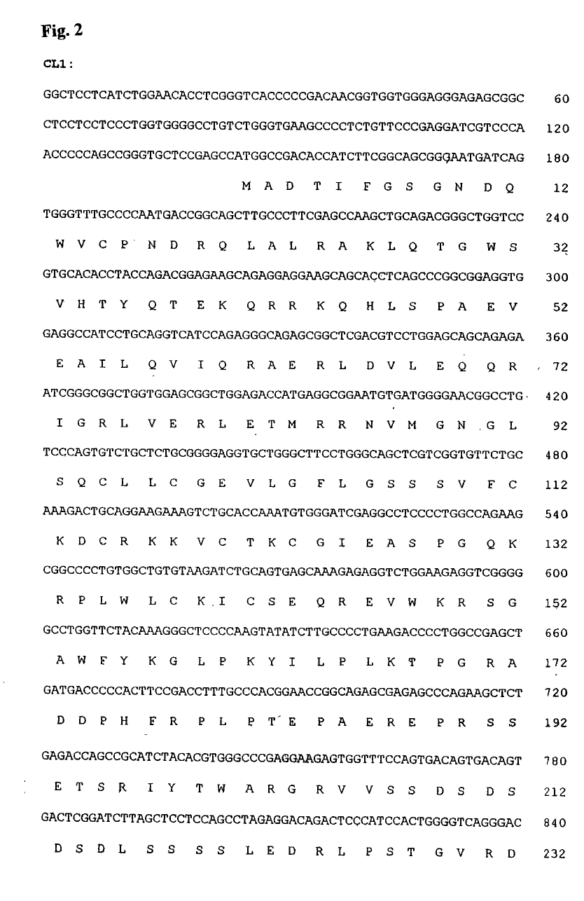

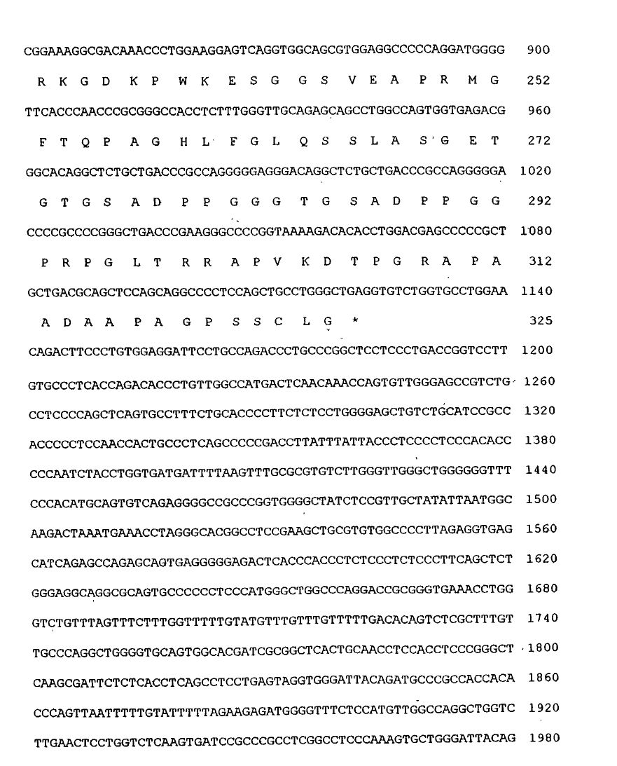

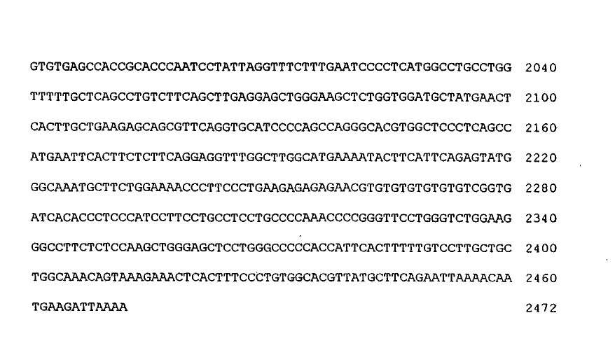

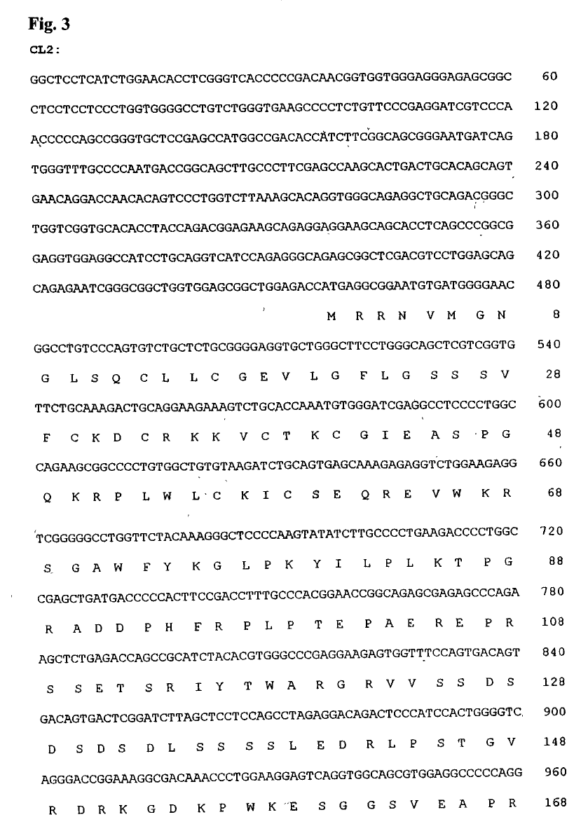

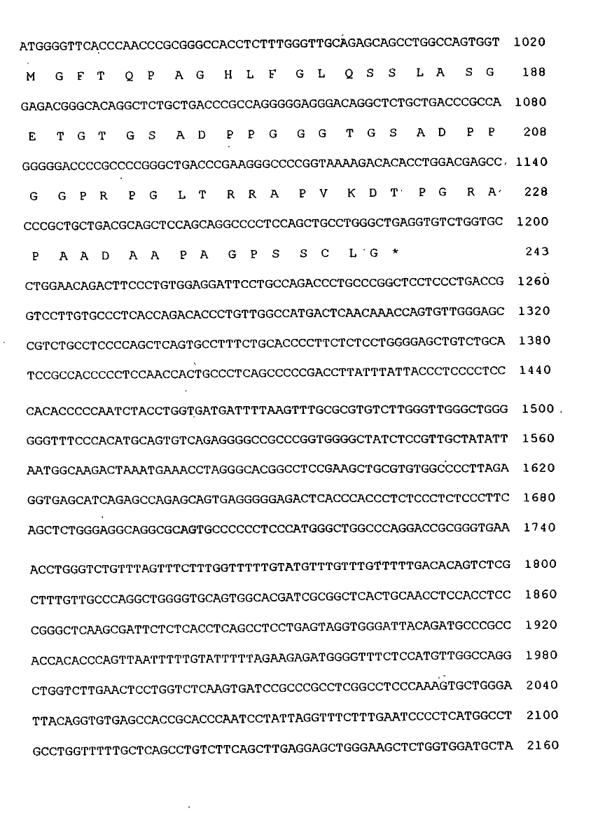

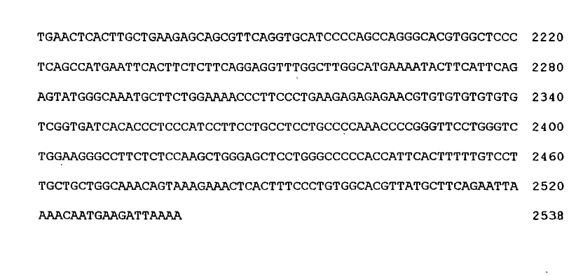

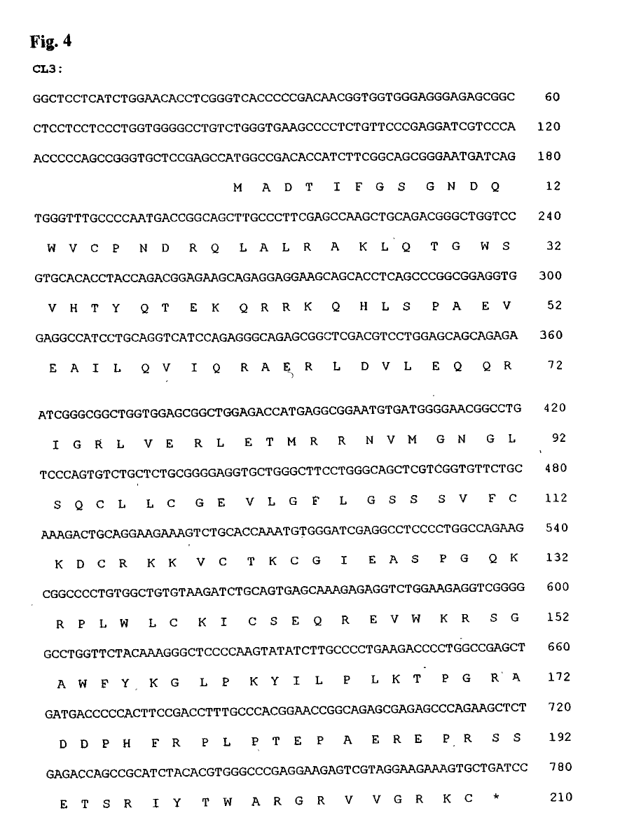

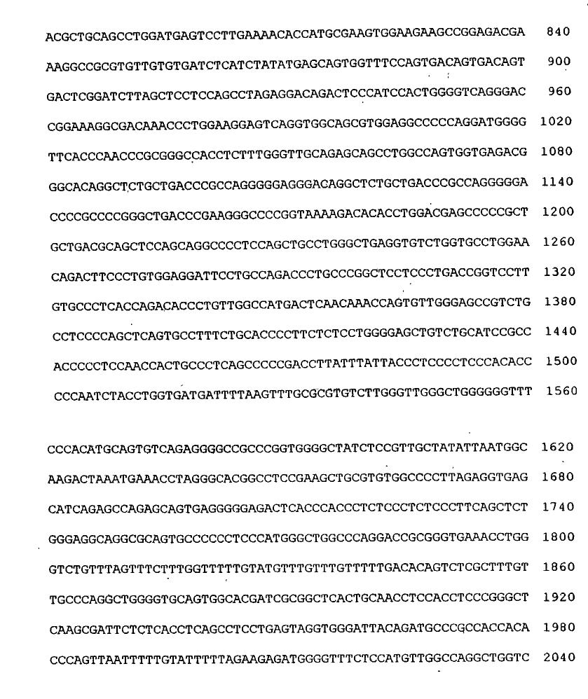

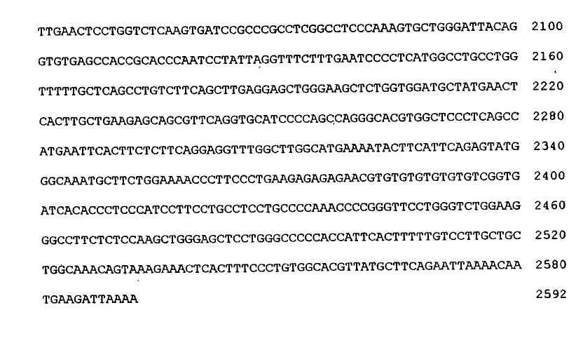

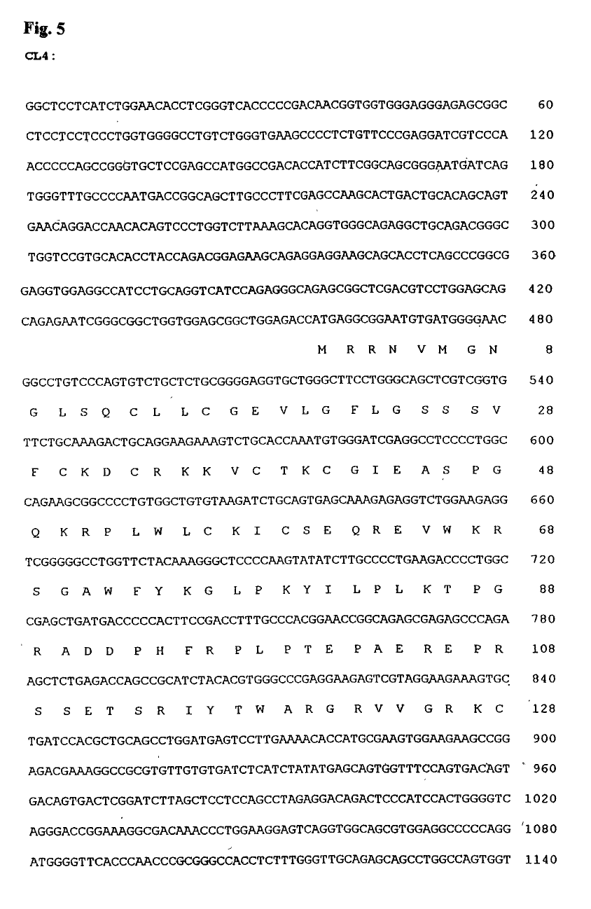

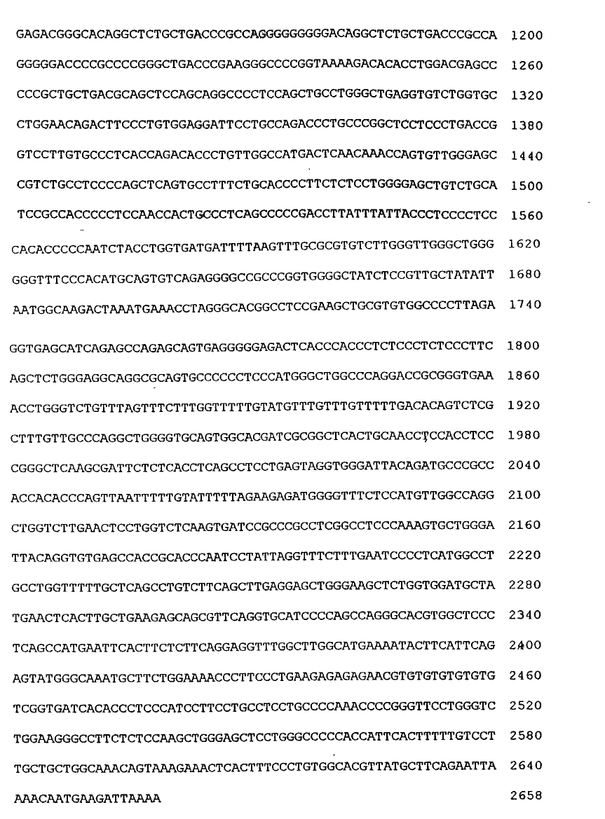

According to the present invention, human NOC2 cDNA sequence was used to query the human lung EST databases (a normal lung and a large cell lung, cancer) using BLAST program to search for NOC2-related gene variants. Five human cDNA clones with partial sequences (i.e., ESTs) deposited in the databases showing similar to NOC2 were isolated and sequenced. Of these clones, one (named NL1) was from normal lung cDNA library and the rest four (named LC1, LC2, LC3, and LC4) were from large cell lung cancer cDNA library. FIGS. 1 to 5 show the nucleic acid sequences (SEQ ID NOs:1, 3, 5, 7, and 9) of the variants and corresponding amino acid sequences (SEQ ID NOs:2, 4, 6, 8, and 10) encoded thereby.

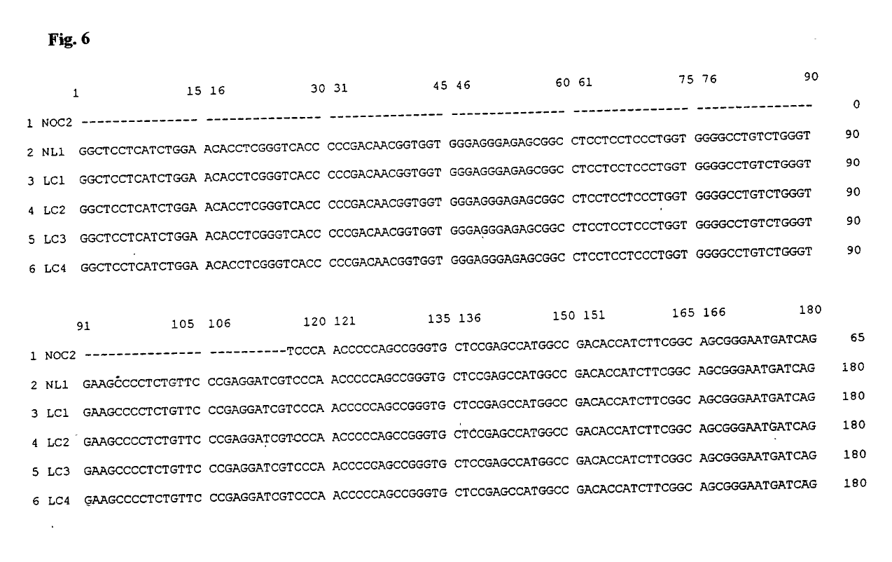

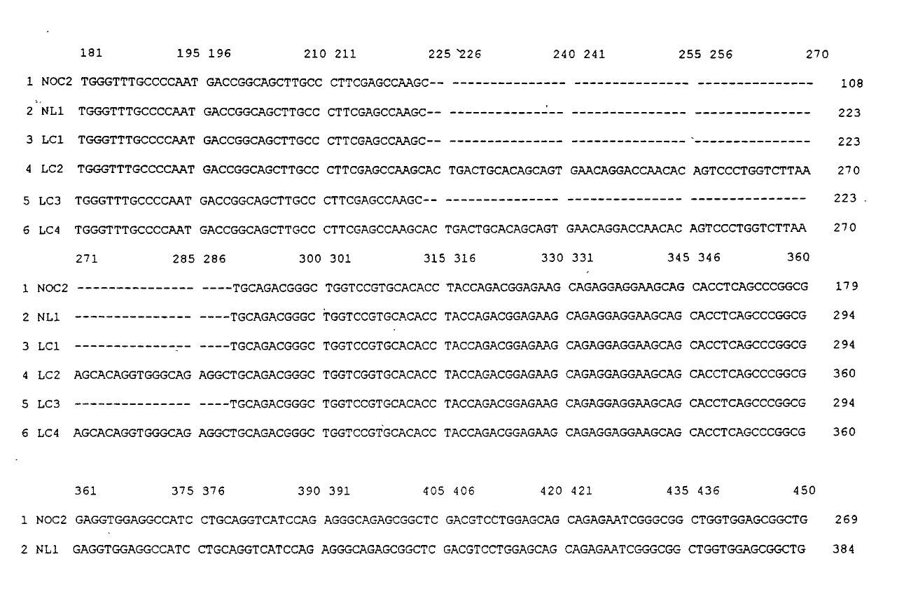

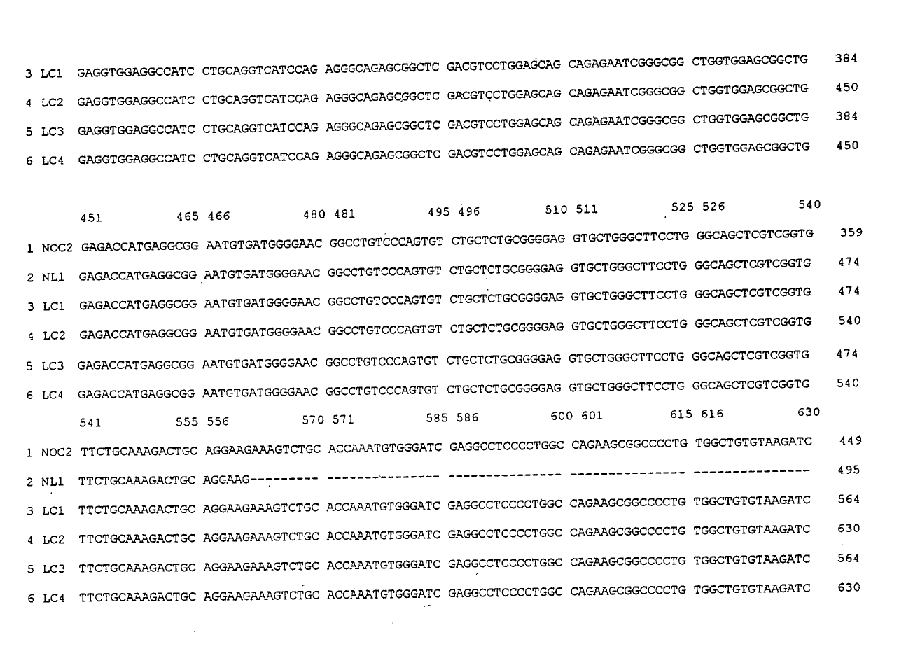

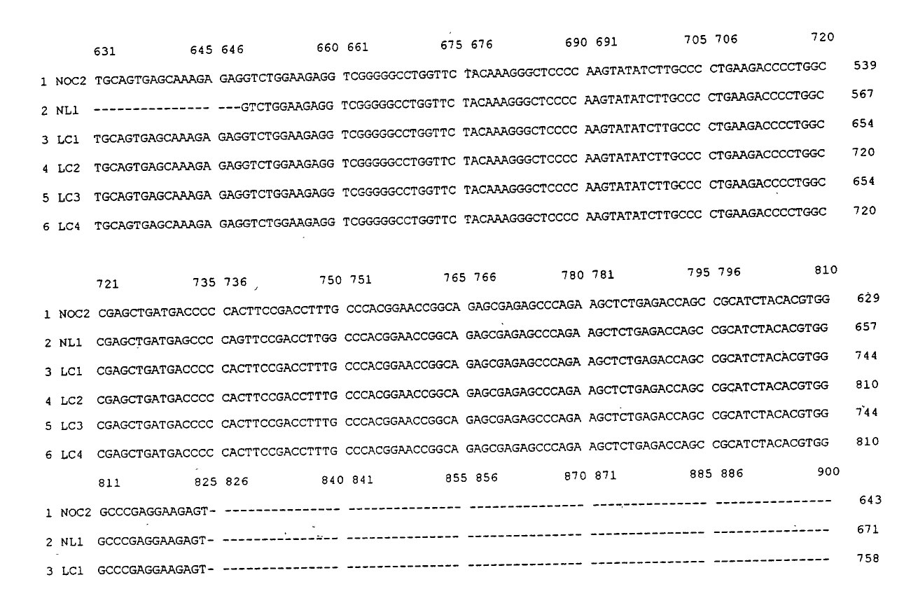

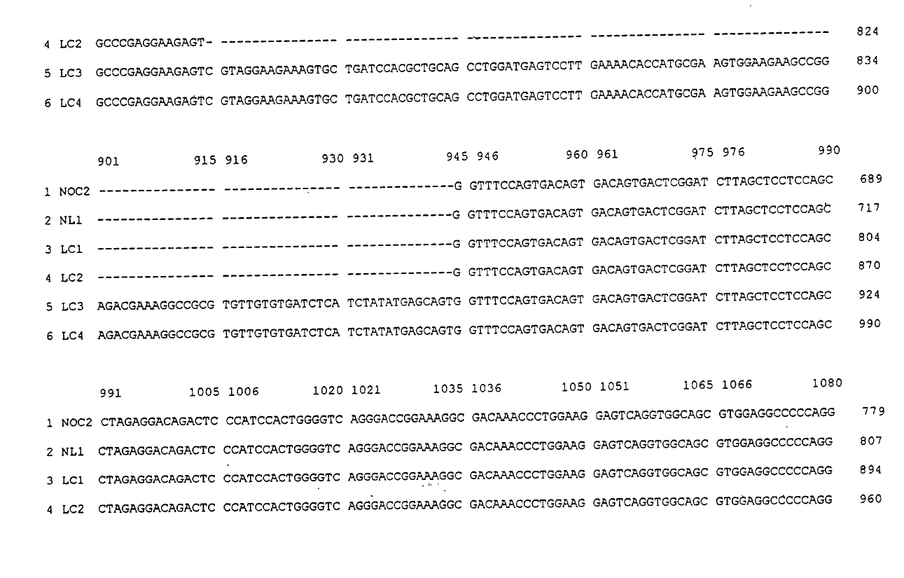

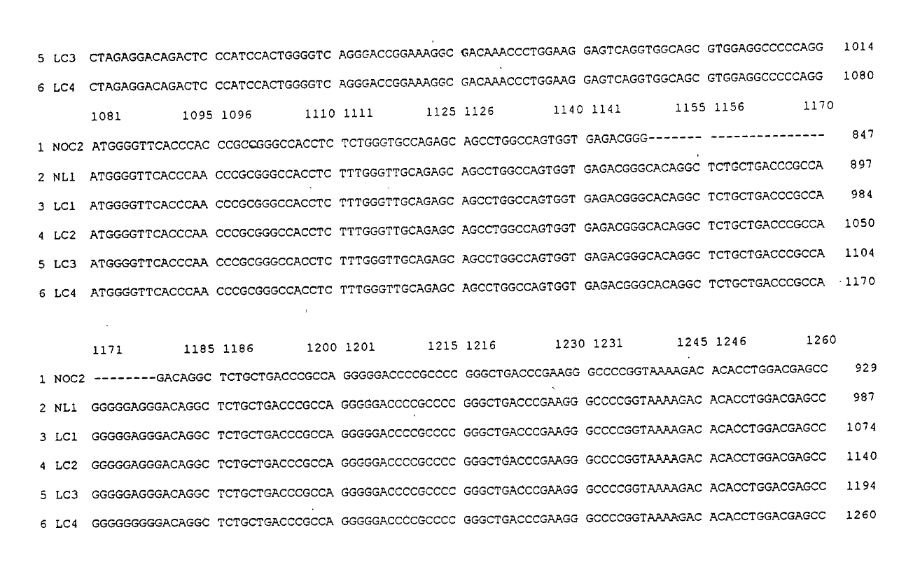

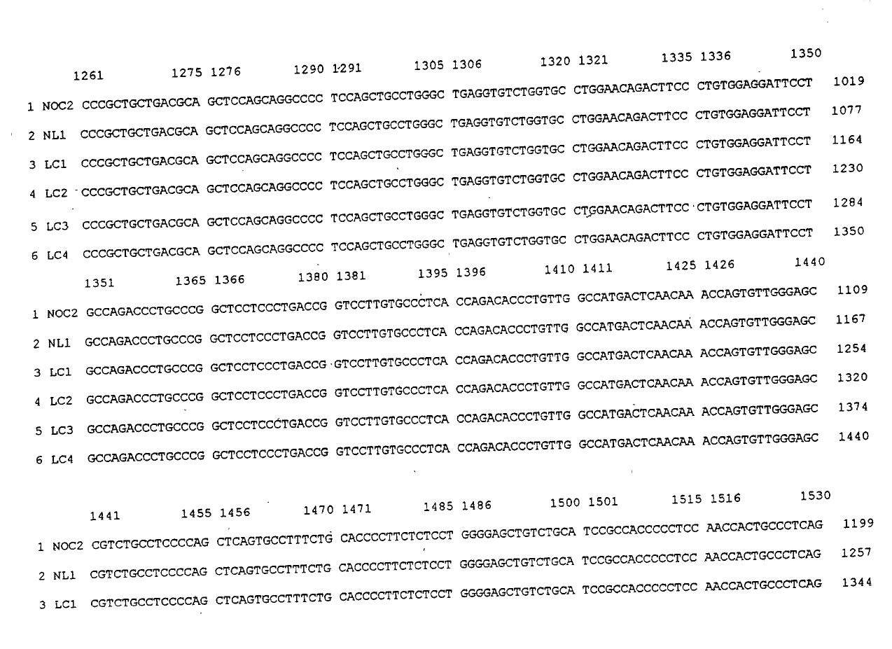

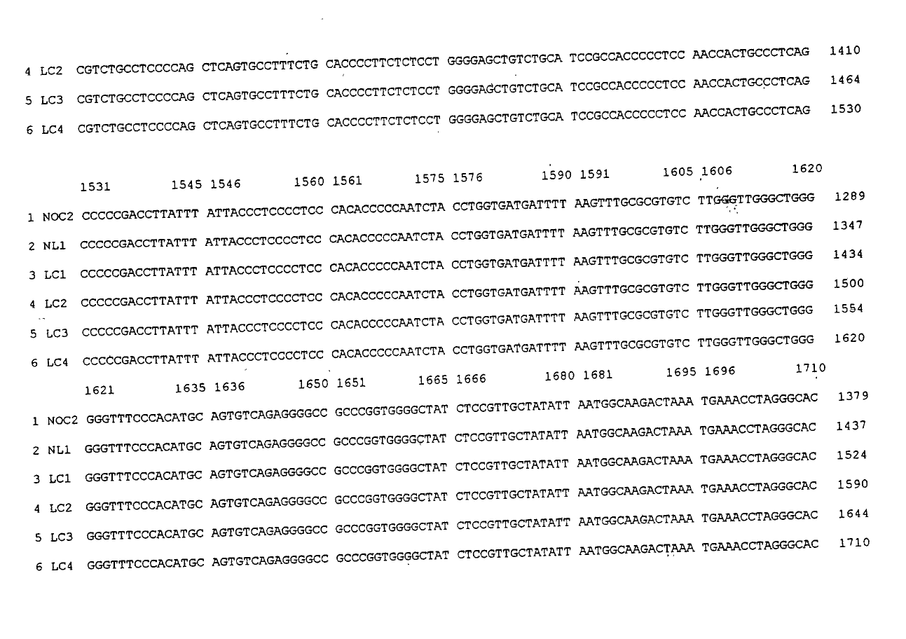

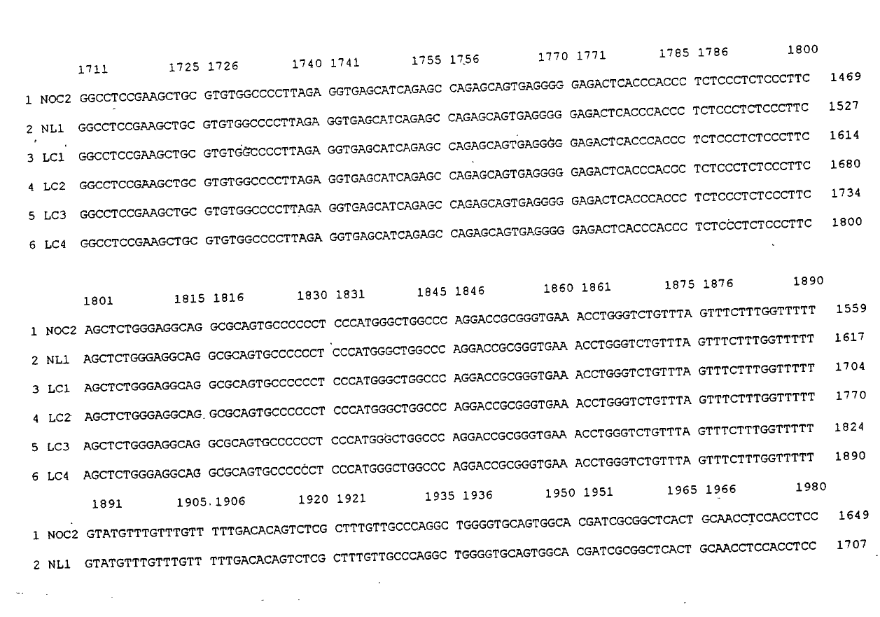

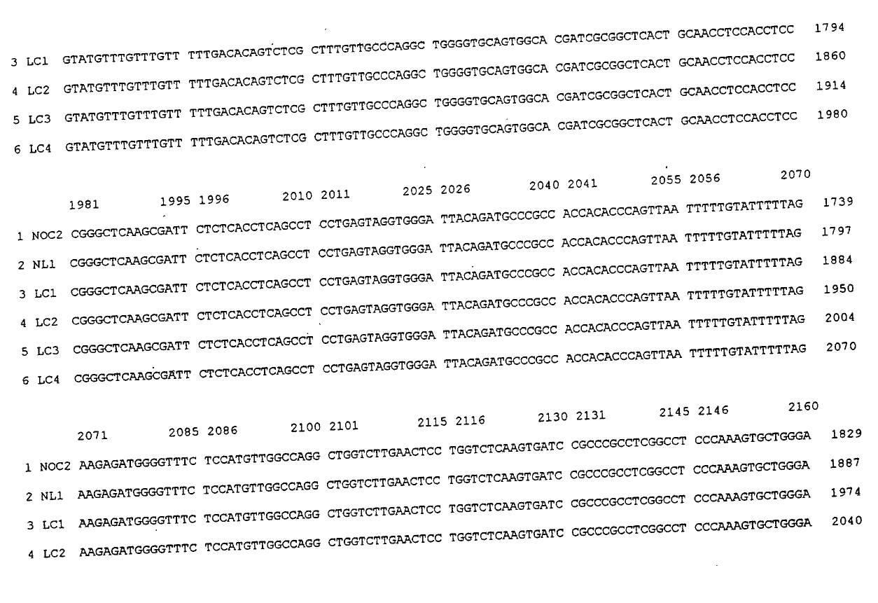

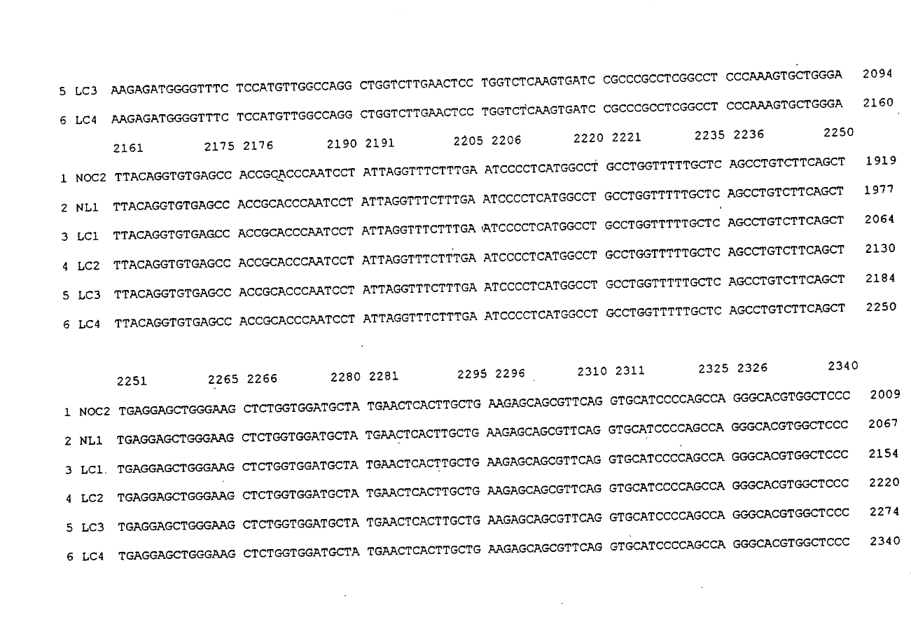

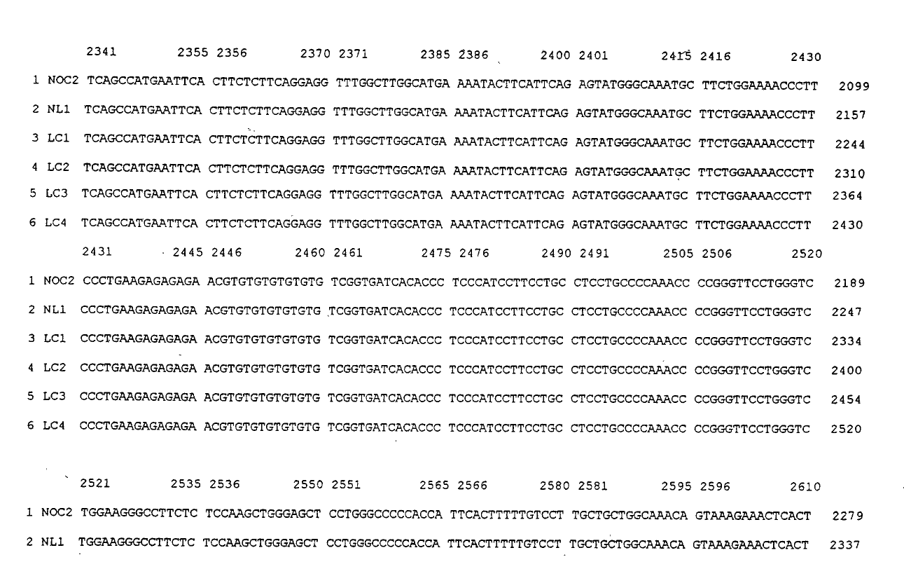

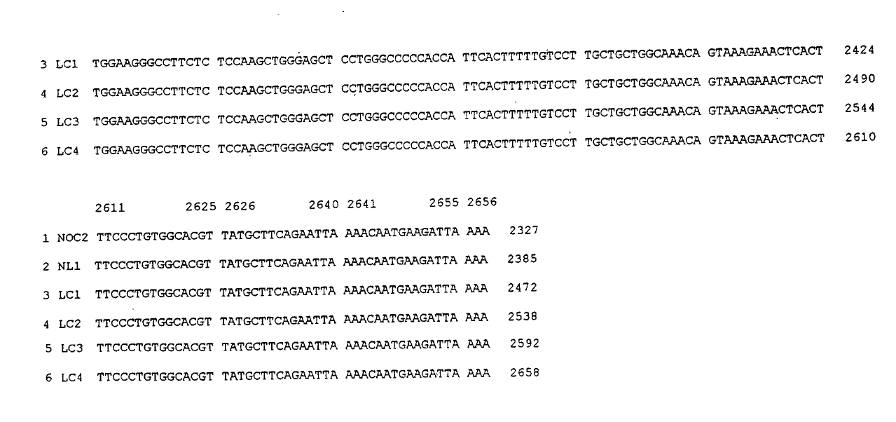

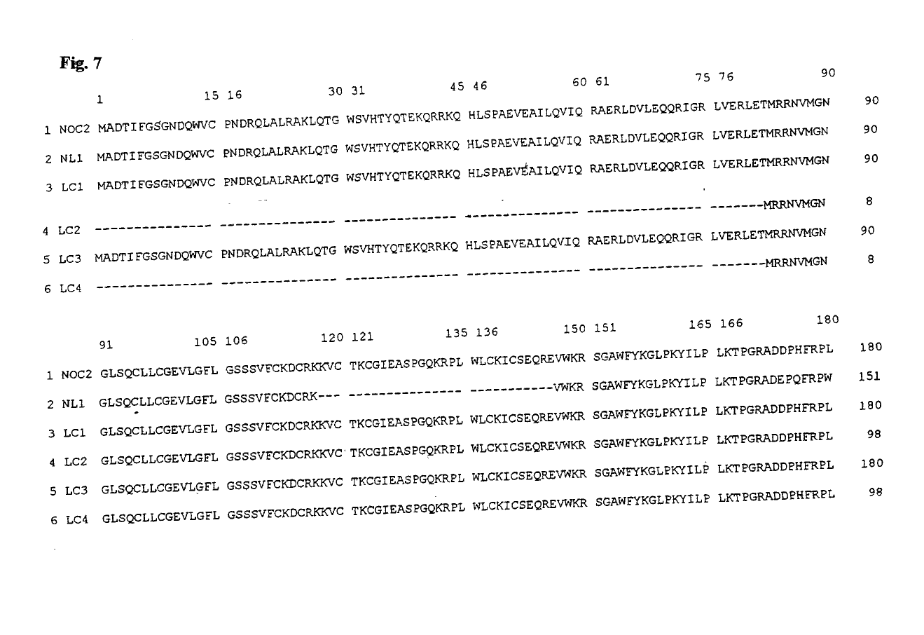

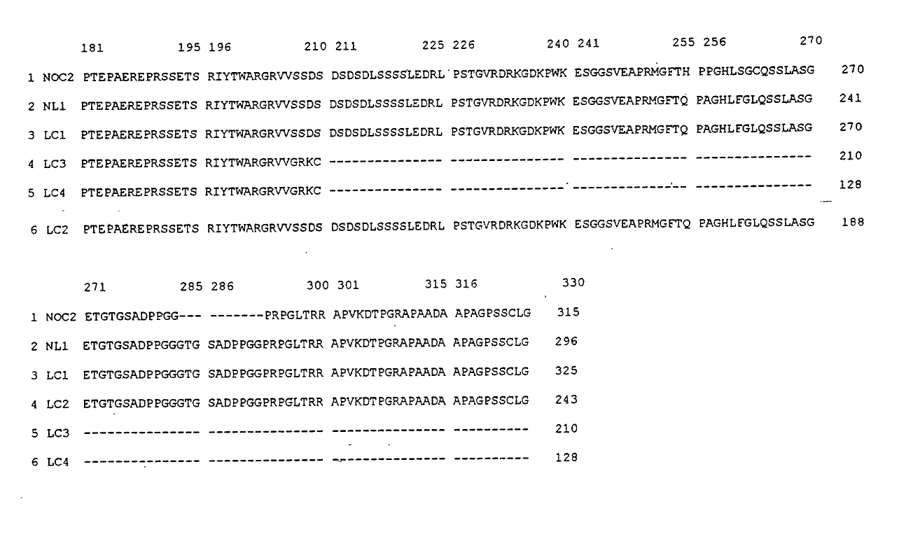

The full-length of the NL1 cDNA is a 2385 bp clone containing an 888 bp open reading frame (ORF) extending from 145-1032 bp, which corresponds to an encoded protein of 296 amino acid residues with a predicted molecular mass of 32.1 kDa. The full-length of the LC1 cDNA is a 2472 bp clone containing a 975 bp ORF extending from 145 to 1119 bp, which corresponds to an encoded protein of 325 amino acid residues with a predicted molecular mass of 35.3 kDa. The full-length of the LC2 cDNA is a 2538 bp clone containing a 729 bp ORF extending from 457 to 1185 bp, which corresponds to an encoded protein of 243 amino acid residues with a predicted molecular mass of 25.8 kDa. The full-length of the LC3 cDNA is a 2592 bp clone containing a 630 bp ORF extending from 145 to 774 bp, which corresponds to an encoded protein of 210 amino acid residues with a predicted molecular mass of 24.1 kDa. The fill-length of the LC4 cDNA is a 2658 bp clone containing a 384 bp ORF extending from 457 to 840 bp, which corresponds to an encoded protein of 128 amino acid residues with a predicted molecular mass of 14.5 k/Da. The sequences around the initiation ATG codon of NL1, LC1 and LC3 (located at nucleotide 145 to 147 bp) and of LC2 and LC4 (located at nucleotide 457 to 459 bp) were matched with the Kozak consensus sequence (A/GCCATGG) (Kozak, (1987) Nucleic Acids Res. 15: 8125-48; Kozak, (1991) J Cell Biol. 115: 887-903.). To determine the variations (insertion/deletion) in sequences of NL1 and LC1-LC4 cDNA clones, an alignment of NOC2 nucleotide/amino acid sequence with these clones was performed (FIGS. 6 and 7). The results indicate that two major genetic alterations were found in the aligned sequences.

The first difference is that the matched nucleotide sequence starts from 220 bp of NOC2 and 116 bp of all clones (NL1 and LC1-LC4). This indicates that the 5′-UTR sequences between NOC2 and, the five isolated cDNA clones are different. The possible explanations for the presence of 5′-UTR sequence variants are: 1) they may have different effects on translation; and/or 2) they may be associated with different tissue distribution since it has been reported that the presence of multiple 5′-UTR variants in the bovine growth hormone receptor mRNA is associated with the gene tissue distributions (Jiang and Lucy, (2001) Gene. 265: 45-53).

The second difference is that several in-frame sequence variations (insertion or splicing) in the coding regions of our clones were found as compared to the NOC2 sequence. For example, 1) an additional 66 bp (22aa) insert was found in sequences of LC2 and LC4 from 224 to 289 bp; 2) a 87 bp (29aa) segment was spliced out in sequence of NL1 at 495 bp; 3) an additional 120 bp (40aa) insert was found in sequence of LC3 from 759 to 878 bp and LC4 from 825 to 944 bp; and 4) an additional 30 bp (10aa) insert was found in sequences of NL1 from 876 to 905 bp, LC1 from 963 to 992 bp, LC2 from 1029 to 1058 bp, LC3 from 1083 to 1112 bp, and LC4, from 1149 to 1178 bp.

In the present invention, a search of ESTs deposited in dbEST (Boguski et al., (1993) nat Genet. 4: 332-3) at NCB1 was performed. ESTs matched to the sequence fragments that contain genetic changes (insertion or splicing) were identified. For example, an EST (GenBank accession number BG33 1517) confirmed the 66 bp insert at 224 to 289 bp of LC2 and LC4. An EST (GenBank accession number BG506767) confirmed the alternative spliced 87 bp at 495 bp of NL1. Two ESTs (GenBank accession number BG331081 and BG332902) confined the 120 bp insert at 759 to 878 bp of LC3 and 825 to 944 bp of LC4. An EST (GenBank accession number BG33 1081) confirmed 30 bp insert at 876 to 905 bp of NL1, 963 to 992 bp of LC1, 1029 to 1058 bp of LC2, 1083 to 1112 bp of LC3, and 1149 to 1178 bp of LC4. The subject invention surprisingly found that these ESTs were found only from cDNA libraries derived from normal lung or large cell lung cancer tissues. This suggests that these nucleotide fragments are important in association with NSCLC, in particular, the large cell lung cancer.

Scanning the NOC2 sequence against the profile entries in PROSITE has indicated that NOC2 protein contains a FYVE zinc finger domain at the position of 89 to 146aa and a serine-rich region at the position of 207 to 220aa. A search of the predicted protein products of NL1 and LC1-LC4 against the profile entries in PROSITE showed that many variations exist in the sequence of FYVE zinc finger domain and serine-rich region. For example, NL1 protein only contains a serine-rich region at position 178 to 191aa. Both LC1 and LC2 proteins contain a FYVE zinc finger domain (89 to 146aa and 7 to 64aa) and a serine-rich region (207 to 220aa and 125 to 138aa). LC3 and LC4 protein contain only a FYVE zinc finger domain at the position of 89 to 146aa and 7 to 64aa, respectively.

Other putative conserved features identified in 1) NOC2 include four protein kinase C phosphorylation sites (38 to 40, 82 to 84, 194 to 196, and 288 to 290aa), five casein kinase II phosphorylation sites (8 to 11, 48 to 51, 208 to 211, 210 to 213, and 212 to 215aa), four N-mynistoylation site (89 to 94, 91 to 96, 262 to 267, and 273 to 278aa), and one gram-positive cocci surface proteins anchoring hexapeptide (225 to 230aa); 2) NL1 include four protein kinase C phosphorylation sites (38 to 40, 82 to 84, 165 to 167, and 269 to 271aa), five casein kinase II phosphorylation sites (8 to 11, 48 to 51, 179 to 182, 181 to 184, and 183 to 186aa), seven N-myristoylation site (89 to 94, 91 to 96, 233 to 238, 244 to 249, 252 to 257, 253 to 258, and 254 to 259aa), and one gram-positive cocci surface proteins anchoring hexapeptide (196 to 201 aa); 3) LC1 include four protein kinase C phosphorylation sites (38 to 40, 82 to 84, 194 to 196, and 298 to 300aa), five casein kinase 11 phosphorylation sites (8 to 11, 48 to 51, 208 to 211, 210 to 213, 212 to 215aa), seven N-myristoylation site (89 to 94, 91 to 96, 262 to 267, 273 to 278, 283 to 288, 281 to 286, and 282 to 287aa), and one gram-positive cocci surface proteins anchoring hexapeptide (225 to 230aa); 4) LC2 include two protein kinase C phosphorylation sites (112 to 114, and 216 to 218aa), three casein kinase II phosphorylation sites (126 to 129, 128 to 131, and 130 to 133aa), seven N-myristoylation site (7 to 12, 9 to 14, 180 to 185, 191 to 196, 199 to 204, 200 to 205, and 201 to 206aa), and one gram-positive cocci surface proteins anchoring hexapeptide (143 to 148aa); 5) LC3 include three protein kinase C phosphorylation sites (38 to 40, 82 to 84, and 194 to 196aa), two casein kinase 11 phosphorylation sites (8 to 11, and 48 to 51aa), two N-myristoylation site (89 to 94, and 91 to 96aa), and one amidation site (206 to 209aa); 6) LC4 include one protein kinase C phosphorylation sites (112 to 114aa), two N-myristoylation site (7 to 12, and 9 to 14aa), and one amidation site (124 to 127aa). In the case of this invention, partial or complete deletion of the FYVE zinc finger domain or serine-rich region of NOC2 protein may result in the protein with truncated or deleted functional domain, suggesting that the functional role of these NOC2-related gene variants may not be the same as NOC2.

According to the present invention, the polypeptides of the human NOC2-related gene variants and fragments thereof may be produced, through genetic engineering techniques. In this case, they are produced by appropriate host cells that has been transformed by DNAs that code for the polypeptides or fragments thereof. The nucleotide sequence encoding the polypeptide of the human NOC2-related gene variants or fragment thereof is inserted into an appropriate expression vector, i.e., a vector which contains the necessary elements for the transcription and translation of the inserted coding sequence in a suitable host. The nucleic acid sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing sequences encoding the polypeptides of the human NOC2-related gene variants and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but not limited to, microorganisms such as bacteria transformed with recombinant bacteriophage, plasmid, or cosmid DNu expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with viruss (e.g., vaccma virus, adenovirus, etc.). Preferably, the host cell is a bacterium, and most preferably, the bacterium is E. coli.

Alternatively, the Polypeptides of the human NOC2-related gene variants or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 4311A peptide synthesizer (Perkin-Elmer).

According to the present invention, the fragments of the polypeptides and nucleic acid sequences of the human NOC2-related gene variants are used as immunogens and primers or probes, respectively. Preferable, the purified fragments of the human NOC2-related gene variants are used. The fragments may be produced by enzyme digestion, chemical cleavage of isolated or purified polypeptide or nucleic acid sequences, or chemical synthesis and then may be isolated or purified. Such isolated or purified fragments of the polypeptides and nucleic acid sequences can be used directed as immunogens and primers or probes, respectively.

The present invention further provides the antibodies which specifically bind one or more out-surface epitopes of the polypeptides of the human NOC2-related gene variants.

According to the present invention, immunization of mammals with immunogens described herein, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and U.S. Pat. No. 4,196,265. Briefly, an animal is immunized with the immunogen. Hybridomas are prepared by fusing spleen cells from the immunized animal with myeloma cells. The fusion products are screened for those, producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)2 fragments.

The subject invention also provides methods for diagnosing the diseases associated with the gene variants of the invention or NSCLC, more preferably, the large cell lung cancer, by the utilization of the nucleic acid sequences, the polypeptide of the human NOC2-related gene variants, or fragments thereof, and the antibodies against the polypeptides.

Many gene variants have been found to be associated with diseases (Stallings-Mann et al., (1996) Proc Natl Acad Sci USA 93: 12394-9; Liu et al., (1997) Nat Genet 16:328-9; Siffert et al., (1998) Nat Genet 18: 45 to 8; Lukas et al., (2001) Cancer Res 61: 3212 to 9). Since NOC2, a putative tumor suppressor gene, is associated with a region (chromosome 17p) of frequent loss of heterozygosity in NSCLC, it is advisable that the gene variants of the present invention, which have genetic changes (insertion or deletion of nucleotide/amino acid sequences) of tumor suppressor genes, may result in cancer development and be useful as markers for the diagnosis of human lung cancer. Based on the cDNA libraries, these NOC2-related gene variants were classified into NSCLC associated NOC2-related gene variants (LC1, LC2, LC3 and LC4) and normal lung associated NOC2-related gene variant (NL1). Thus, the expression level of NSCLC associated NOC2-related gene variants relative to normal lung associated NOC2-related gene variant may be a useful indicator for screening of patients suspected of having NSCLC. This suggests that the index of relative expression level (mRNA or protein) may confer an increased susceptibility to NSCLC, more preferably, the large cell lung cancer. Fragments of NOC2-related gene variant transcripts (mRNAs) may be detected by RT-PCR approach. Polypeptides of NOC2-related gene variants may be determined by the binding of antibodies to these polypeptides. These approaches may be performed in accordance with conventional methods well known by persons skilled in the art.

According to the present invention, the expression of these gene variant mRNAs in sample may be determined by, but not limited to, RT-PCR. Using TRIZOL reagents (Life Technology), total RNA may be isolated from patient samples. Tissue samples (e.g., biopsy samples) are powdered under liquid nitrogen before homogenization. RNA purity and integrity are assessed by absorbance at 260/280 nm and by agarose gel electrophoresis. Two sets of primers, such as one set for NL1 and the other set for any NSCLC associated NOC2-related gene variants (e.g., LC1 to LC4), are designed to co-amplify the expected sizes of specific PCR fragments of gene variants. PCR fragments are analyzed on a 1% agarose gel using five microliters (10%) of the amplified products. The intensity of the signals may be determined by using the Molecular Analyst program (version 1.4.1; Bio-Rad). Thus, the index of relative expression levels for each co-amplified PCR products may be calculated based on the intensity of signals.

The RT-PCR experiment may be performed according to the manufacturer instructions (Boeluinger Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 p 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

The expression of gene variants can also be analyzed using Northern Blot hybridization approach. Specific fragments of the gene variants may be amplified by polymerase chain reaction (PCR). The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the presence of the polypeptides of these gene variants in samples may be determined by, but not limited to, the immunoassay which uses the antibodies specifically binding to the polypeptides. The polypeptides of the gene variants may be expressed in prokaryotic cells by using suitable prokaryotic expression vectors. The cDNA fragments of NL1 and LC1-LC4 gene encoding the amino acid coding sequence may be PCR amplified with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 h. The bacterially-expressed proteins may be purified.

The polypeptides of the gene variants may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated 4 in 12-well plates one day before transfection at a density of 5×10⁴ cells per well. Transfections may be carried out using Lipofectamine Plus transfection reagent according to the manufacturer's instructions (Gibco BRL). Three hours for flowing transfection, medium containing the complexes may be replaced with fresh medium. Forty-eight hours after incubation, the cells may be scraped into lysis buffer (0.1 M Tris HCl, pH, 8.0, 0.1% Triton X-100) for purification of expressed proteins. After these proteins are purified, monoclonal antibodies against these purified proteins (NL1, LC1-LC4) may be generated using hybridoma technique according to the conventional methods (de StGroth and Scheidegger, (1980) J finunol Methods 35:1-21; Cote et al. (1983) Proc Natl Acad Sci USA 80: 2026-30; and Kozbor et al. (1985) J Immunol Methods 81:31-42).

According to the present invention, the presence of the polypeptides of the gene variants in samples of normal lung and lung cancers may be determined by, but not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 m-M glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptides of gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal and large cell 15 lung cancer) cDNA clones were compiled and served as EST databases.

Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative NOC2 encoding gene were identified during the course of EST generation.

### Isolation of cDNA Clones

Five cDNA clones exhibiting EST sequences similar to the NOC2 gene were isolated from the lung cDNA libraries and named NL1 (from normal lung) and LC1 to LC4 (from large cell lung cancer). The inserts of these clones were subsequently excised in vivo from the KZAP Express vector using the ExAssist/XLOLR helper phage system (Stratagene). Phagemid particles were excised by coinfecticg XL1-BLUE MRF' cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Taq Dyedeoxy Terminator Cycle Sequencing Kit for Applied Biosystems 377 sequencing system (Perkin Elmer). Using the primer-walking approach, full-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

What is claimed is:

1. An isolated polypeptide comprising the amino acid sequence of SEQ ID NO: 2, 4, 6, 8, or 10, and fragments thereof.

2. The isolated polypeptide of claim 1, wherein the fragments comprise the amino acid residues 1 to 82 or 254 to 264 of SEQ ID NO: 2.

3. The isolated polypeptide of claim 1, wherein the fragments comprise the amino acid residues 1 to 82, 118 to 146, or 283 to 292 of SEQ ID NO: 4.

4. The isolated polypeptide of claim 1, wherein the fragments comprise the amino acid residues 36 to 64 or 201 to 211 of SEQ ID NO: 6.

5. The isolated polypeptide of claim 1, wherein the fragments comprise the amino acid residues 1 to 82 or 118 to 146 of SEQ ID NO: 8.

6. The isolated polypeptide of claim 1, wherein the fragments comprise the amino acid residues 36 to 64 of SEQ ID NO: 10.

7. An isolated nucleic acid encoding the polypeptide of any of claims 1 to 6, and fragments thereof.

8. The isolated nucleic acid of claim 7, which is the nucleotide sequence of SEQ ID NO: 1, 3, 5, 7, or 9.

9. The isolated nucleic acid of claim 7, wherein the fragments comprise the nucleotides 1 to 115 bp or 876 to 905 bp of SEQ ID NO: 1.

10. The isolated nucleic acid of claim 7, wherein the fragments comprise the nucleotides 1 to 115 bp, 224 to 289 bp, or 963 to 992 bp of SEQ ID NO: 3.

11. The isolated nucleic acid of claim 7, wherein the fragments comprise the nucleotides 1 to 115 bp, 495 to 582 bp, 561 to 648 bp, or 1029 to 1058 bp of SEQ ID NO: 5.

12. The isolated nucleic acid of claim 7, wherein the fragments comprise the nucleotides 1 to 115 bp, 495 to 582 bp, 759 to 878 bp, or 1083 to 1112 bp of SEQ ID NO: 7.

13. The isolated nucleic acid of claim 7, wherein the fragments comprise the nucleotides 1 to 115 bp, 224 to 289 bp, 561 to 648 bp, 825 to 944 bp, or 1149 to 1178 bp of SEQ ID NO: 9.

14. An expression vector comprising the nucleic acid of any one of claims 7 to 13.

15. A host cell comprising the expression vector of claim 14.

16. A method for producing the polypeptide of any one of claims 1 to 6, which comprises the steps of:
(1) culturing the host cell of claim 15 under a condition suitable for the expression of the polypeptide; and
(2) recovering the polypeptide from the host cell culture.

17. An antibody specifically binding to the polypeptide of any one of claims 1 to 6.

18. The antibody of claim 17 is a polyclonal or monoclonal antibody.

19. A method for detecting the presence of the nucleic acid of any one of claims 7 to 13 in a mammal, which comprises the steps of:
(1) extracting total RNA from a sample obtained from the mammal;
(2) amplifying the RNA by reverse transcriptase-polymerase chain reaction (RT-PCR) to obtain a cDNA sample;
(3) hybridizing the cDNA sample with the nucleic acid of any one of claims 7 to 13; and
(4) detecting the amount of the hybridized sample.

20. The method of claim 17, wherein the hybridizing process is conducted by Northern blot approach or microarray approach.

21. The method of claim 19, which is useful in diagnosing non-small cell lung cancer.

22. The method of claim 21, wherein the non-small cell lung cancer is large cell lung cancer.

23. A method for detecting the presence of the polypeptide of any one of claims 1 to 6 in a mammal, which comprises the steps of contacting the antibody of claim 17 or 18 with protein samples extracting from the mammal, and detecting the amount of antibody-antigen binding samples.

24. The method of claim 23, wherein the antibody-antigen binding samples are detected by Western blot approach.

25. The method of claim 23, which is useful in diagnosing non-small cell lung cancer.

26. The method of claim 25, wherein the non-small cell lung cancer is large cell lung cancer.


SEQUENCE LISTING (10 sequences)

SEQ ID NO: 1 - DNA, 2385 bp, HOMO SAPIEN
  CDS (145)..(1032)
ggctcctcatctggaacacctcgggtcacccccgacaacggtggtgggagggagagcggcctcctcctccctggtggggcctgtctgggtgaagcccctctgttcccgaggatcgtcccaacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaaggtctggaagaggtcgggggcctggttctacaaagggctccccaagtatatcttgcccctgaagacccctggccgagctgatgagccccagttccgaccttggcccacggaaccggcagagcgagagcccagaagctctgagaccagccgcatctacacgtgggcccgaggaagagtggtttccagtgacagtgacagtgactcggatcttagctcctccagcctagaggacagactcccatccactggggtcagggaccggaaaggcgacaaaccctggaaggagtcaggtggcagcgtggaggcccccaggatggggttcacccaacccgcgggccacctctttgggttgcagagcagcctggccagtggtgagacgggcacaggctctgctgacccgccagggggagggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccatgggctggcccaggaccgcgggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgctcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgatcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaa

SEQ ID NO: 2 - protein, 296 aa, HOMO SAPIEN
MADTIFGSGNDQWVCPNDRQLALRAKLQTGWSVHTYQTEKQRRKQHLSPAEVEAILQVIQRAERLDVLEQQRIGRLVERLETMRRNVMGNGLSQCLLCGEVLGFLGSSSVFCKDCRKVWKRSGAWFYKGLPKYILPLKTPGRADEPQFRPWPTEPAEREPRSSETSRIYTWARGRVVSSDSDSDSDLSSSSLEDRLPSTGVRDRKGDKPWKESGGSVEAPRMGFTQPAGHLFGLQSSLASGETGTGSADPPGGGTGSADPPGGPRPGLTRRAPVKDTPGRAPAADAAPAGPSSCLG

SEQ ID NO: 3 - DNA, 2472 bp, HOMO SAPIEN
  CDS (145)..(1119)
ggctcctcatctggaacacctcgggtcacccccgacaacggtggtgggagggagagcggcctcctcctccctggtggggcctgtctgggtgaagcccctctgttcccgaggatcgtcccaacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgacagtgactcggatcttagctcctccagcctagaggacagactcccatccactggggtcagggaccggaaaggcgacaaaccctggaaggagtcaggtggcagcgtggaggcccccaggatggggttcacccaacccgcgggccacctctttgggttgcagagcagcctggccagtggtgagacgggcacaggctctgctgacccgccagggggagggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccatgggctggcccaggaccgcgggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgctcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgatcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaa

SEQ ID NO: 4 - protein, 325 aa, HOMO SAPIEN
MADTIFGSGNDQWVCPNDRQLALRAKLQTGWSVHTYQTEKQRRKQHLSPAEVEAILQVIQRAERLDVLEQQRIGRLVERLETMRRNVMGNGLSQCLLCGEVLGFLGSSSVFCKDCRKKVCTKCGIEASPGQKRPLWLCKICSEQREVWKRSGAWFYKGLPKYILPLKTPGRADDPHFRPLPTEPAEREPRSSETSRIYTWARGRVVSSDSDSDSDLSSSSLEDRLPSTGVRDRKGDKPWKESGGSVEAPRMGFTQPAGHLFGLQSSLASGETGTGSADPPGGGTGSADPPGGPRPGLTRRAPVKDTPGRAPAADAAPAGPSSCLG

SEQ ID NO: 5 - DNA, 2538 bp, HOMO SAPIEN
  CDS (457)..(1185)
ggctcctcatctggaacacctcgggtcacccccgacaacggtggtgggagggagagcggcctcctcctccctggtggggcctgtctgggtgaagcccctctgttcccgaggatcgtcccaacccccagccgggtgctccgagccatggccgacaccatcttcggcagcgggaatgatcagtgggtttgccccaatgaccggcagcttgcccttcgagccaagcactgactgcacagcagtgaacaggaccaacacagtccctggtcttaaagcacaggtgggcagaggctgcagacgggctggtcggtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtggtttccagtgacagtgacagtgactcggatcttagctcctccagcctagaggacagactcccatccactggggtcagggaccggaaaggcgacaaaccctggaaggagtcaggtggcagcgtggaggcccccaggatggggttcacccaacccgcgggccacctctttgggttgcagagcagcctggccagtggtgagacgggcacaggctctgctgacccgccagggggagggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccatgggctggcccaggaccgcgggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgctcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgatcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaa

SEQ ID NO: 6 - protein, 243 aa, HOMO SAPIEN
MRRNVMGNGLSQCLLCGEVLGFLGSSSVFCKDCRKKVCTKCGIEASPGQKRPLWLCKICSEQREVWKRSGAWFYKGLPKYILPLKTPGRADDPHFRPLPTEPAEREPRSSETSRIYTWARGRVVSSDSDSDSDLSSSSLEDRLPSTGVRDRKGDKPWKESGGSVEAPRMGFTQPAGHLFGLQSSLASGETGTGSADPPGGGTGSADPPGGPRPGLTRRAPVKDTPGRAPAADAAPAGPSSCLG

SEQ ID NO: 7 - DNA, 2592 bp, HOMO SAPIEN
  CDS (145)..(774)
ggctcctcatctggaacacctcgggtcacccccgacaacggtggtgggagggagagcggcctcctcctccctggtggggcctgtctgggtgaagcccctctgttcccgaggatcgtcccaacccccagccgggtgctccgagccatggccgacaccatcttcggcagcgggaatgatcagtgggtttgccccaatgaccggcagcttgcccttcgagccaagctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtcgtaggaagaaagtgctgatccacgctgcagcctggatgagtccttgaaaacaccatgcgaagtggaagaagccggagacgaaaggccgcgtgttgtgtgatctcatctatatgagcagtggtttccagtgacagtgacagtgactcggatcttagctcctccagcctagaggacagactcccatccactggggtcagggaccggaaaggcgacaaaccctggaaggagtcaggtggcagcgtggaggcccccaggatggggttcacccaacccgcgggccacctctttgggttgcagagcagcctggccagtggtgagacgggcacaggctctgctgacccgccagggggagggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccatgggctggcccaggaccgcgggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgctcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgatcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaa

SEQ ID NO: 8 - protein, 210 aa, HOMO SAPIEN
MADTIFGSGNDQWVCPNDRQLALRAKLQTGWSVHTYQTEKQRRKQHLSPAEVEAILQVIQRAERLDVLEQQRIGRLVERLETMRRNVMGNGLSQCLLCGEVLGFLGSSSVFCKDCRKKVCTKCGIEASPGQKRPLWLCKICSEQREVWKRSGAWFYKGLPKYILPLKTPGRADDPHFRPLPTEPAEREPRSSETSRIYTWARGRVVGRKC

SEQ ID NO: 9 - DNA, 2658 bp, HOMO SAPIEN
  CDS (457)..(840)
ggctcctcatctggaacacctcgggtcacccccgacaacggtggtgggagggagagcggcctcctcctccctggtggggcctgtctgggtgaagcccctctgttcccgaggatcgtcccaacccccagccgggtgctccgagccatggccgacaccatcttcggcagcgggaatgatcagtgggtttgccccaatgaccggcagcttgcccttcgagccaagcactgactgcacagcagtgaacaggaccaacacagtccctggtcttaaagcacaggtgggcagaggctgcagacgggctggtccgtgcacacctaccagacggagaagcagaggaggaagcagcacctcagcccggcggaggtggaggccatcctgcaggtcatccagagggcagagcggctcgacgtcctggagcagcagagaatcgggcggctggtggagcggctggagaccatgaggcggaatgtgatggggaacggcctgtcccagtgtctgctctgcggggaggtgctgggcttcctgggcagctcgtcggtgttctgcaaagactgcaggaagaaagtctgcaccaaatgtgggatcgaggcctcccctggccagaagcggcccctgtggctgtgtaagatctgcagtgagcaaagagaggtctggaagaggtcgggggcctggttctacaaagggctccccaagtatatcttgcccctgaagacccctggccgagctgatgacccccacttccgacctttgcccacggaaccggcagagcgagagcccagaagctctgagaccagccgcatctacacgtgggcccgaggaagagtcgtaggaagaaagtgctgatccacgctgcagcctggatgagtccttgaaaacaccatgcgaagtggaagaagccggagacgaaaggccgcgtgttgtgtgatctcatctatatgagcagtggtttccagtgacagtgacagtgactcggatcttagctcctccagcctagaggacagactcccatccactggggtcagggaccggaaaggcgacaaaccctggaaggagtcaggtggcagcgtggaggcccccaggatggggttcacccaacccgcgggccacctctttgggttgcagagcagcctggccagtggtgagacgggcacaggctctgctgacccgccagggggggggacaggctctgctgacccgccagggggaccccgccccgggctgacccgaagggccccggtaaaagacacacctggacgagcccccgctgctgacgcagctccagcaggcccctccagctgcctgggctgaggtgtctggtgcctggaacagacttccctgtggaggattcctgccagaccctgcccggctcctccctgaccggtccttgtgccctcaccagacaccctgttggccatgactcaacaaaccagtgttgggagccgtctgcctccccagctcagtgcctttctgcaccccttctctcctggggagctgtctgcatccgccaccccctccaaccactgccctcagcccccgaccttatttattaccctcccctcccacacccccaatctacctggtgatgattttaagtttgcgcgtgtcttgggttgggctggggggtttcccacatgcagtgtcagaggggccgcccggtggggctatctccgttgctatattaatggcaagactaaatgaaacctagggcacggcctccgaagctgcgtgtggccccttagaggtgagcatcagagccagagcagtgagggggagactcacccaccctctccctctcccttcagctctgggaggcaggcgcagtgcccccctcccatgggctggcccaggaccgcgggtgaaacctgggtctgtttagtttctttggtttttgtatgtttgtttgtttttgacacagtctcgctttgttgcccaggctggggtgcagtggcacgatcgcggctcactgcaacctccacctcccgggctcaagcgattctctcacctcagcctcctgagtaggtgggattacagatgcccgccaccacacccagttaatttttgtatttttagaagagatggggtttctccatgttggccaggctggtcttgaactcctggtctcaagtgatccgcccgcctcggcctcccaaagtgctgggattacaggtgtgagccaccgcacccaatcctattaggtttctttgaatcccctcatggcctgcctggtttttgctcagcctgtcttcagcttgaggagctgggaagctctggtggatgctatgaactcacttgctgaagagcagcgttcaggtgcatccccagccagggcacgtggctccctcagccatgaattcacttctcttcaggaggtttggcttggcatgaaaatacttcattcagagtatgggcaaatgcttctggaaaacccttccctgaagagagagaacgtgtgtgtgtgtgtcggtgatcacaccctcccatccttcctgcctcctgccccaaaccccgggttcctgggtctggaagggccttctctccaagctgggagctcctgggcccccaccattcactttttgtccttgctgctggcaaacagtaaagaaactcactttccctgtggcacgttatgcttcagaattaaaacaatgaagattaaaa

SEQ ID NO: 10 - protein, 128 aa, HOMO SAPIEN
MRRNVMGNGLSQCLLCGEVLGFLGSSSVFCKDCRKKVCTKCGIEASPGQKRPLWLCKICSEQREVWKRSGAWFYKGLPKYILPLKTPGRADDPHFRPLPTEPAEREPRSSETSRIYTWARGRVVGRKC